(19) United States
(12) Patent Application Publication
(10) Pub. No.: US 2020/0030051 A1
(43) Pub. Date: Jan. 30, 2020

(54) Health Care Safety Devices

(71) Applicant: Arakel Mfg. Co., Sudbury, MA (US)

(72) Inventor: Aram J. Mirigian, Sudbury, MA (US)

(73) Assignee: Arakel Mfg. Co., Sudbury, MA (US)

(21) Appl. No.: 16/049,023

(22) Filed: Jul. 30, 2018

(51) Int. Cl. A61B 90/00; A61M 5/32; A61B 90/50

(52) U.S. Cl. CPC A61B 90/08; A61M 5/3202; A61B 2090/0801; A61M 2209/084; A61B 90/50

(57) ABSTRACT

The invention relates to a safety device for use by health care professionals to mitigate dangers posed by dirty or contaminated devices such as needles, that is compact, easy to use, and clean.

### BACKGROUND

There is a compelling need in the health care industry for improved safety devices for use by health care professionals (HCP). For example, with alarming frequency doctors and other medical personnel, including physician's assistants, nurses, staff, fire & rescue teams, home care providers and others are potentially risking their lives every day when using needles during the treatment of patients. HCP who accidentally stick themselves with the needles used on patients are in danger of contracting whatever disease that patient may have whether that disease is the reason for the patient's treatment in the first place. HCP may inadvertently stick themselves with a medicament or drug even before treatment of a patient, or inadvertently touch the needle on a contaminated service before treating the patient, and thus placing their lives and patient lives in danger.

Typically, and frequently, these needle sticks involve the use of both hands—one hand holds the needle which then accidentally sticks the other hand with the needle tip. This can occur for many reasons. For example, HCP may be moving too quickly from one place to the other, not paying attention, or even momentarily distracted and then accidentally sticks the other hand. Another frequent source of needle sticks occurs when HCP tries to re-cap or sheath the needle and accidentally sticks the submissive hand.

Accidental needle sticks account for HCP being afflicted with, for example, HIV and Hep C/B to name a few. While such afflictions may be treatable and/or cured, the potential risks are far too high, unacceptable, and 100% preventable. OHSA, CDC, and others have acknowledged these risks and advocated for improved technology and education to reduce needle sticks. See, e.g., Jennifer L. Cleveland DDS, MPH, JADA, “Preventing percutaneous injuries among dental health care personnel”, Vol. 138, February 2007. p. 169-178; Dr. Suneel Kumar Punjabi, “Needle Stick Injuries; Concept & Handling Among Junior Dentist”, Professional Med. J. 2017; 24(1):177-181; Fariba S. Younai, D.D.S., “Occupational Exposures to Blood in a Dental Teaching Environment: Results of a Ten-Year Surveillance Study”. Journal of Dental Education, Vol. 65, No. 5, May 2001, p. 436-448.

### SUMMARY

The present invention provides, among other things, a solution to the problem of accidental needles sticks currently plaguing HCP. The present invention may find applications in other industries as well.

In one embodiment of the invention a sheathed needle in placed in a safety device (“SD”) described herein. HCP using one hand removes the needle from the SD, thereby unsheathing the needle—with only the sheath remaining in the SD. HCP is then able attend to the patient using the unsheathed needle to administer, for example, liquid anesthetic or other required liquid medicament. HCP can then return the un-sheathed needle back into sheath being held in SD. HCP is then able to remove the re-sheathed needle and then discard the needle and sheath pursuant to state and federal regulations, for example in an appropriate sharps container. In the present invention, the SD allows HCP to safely un-sheathe and re-sheathe the needle using only one hand, and thereby substantially reduce the chances of an unintended needle stick potentially creating a serious health issue for HCP.

In one embodiment the SD is a free standing (desk top) device with a weighted base. In another embodiment the SD may be secured to a vertical surface (e.g., wall) in a slightly upward position from horizontal, for example 15+° or higher, to insure the needle and sheath are safely stored in SD when being used and will not accidentally fall out. The SD is safe for use in all HCP environments, including mobile clinics and other compact environments.

The SD of the present invention is easy to use, capable of saving lives of HCP and patients, not to mention saving tax payers and companies thousands if not millions of dollars in medical expenses for HCP, family members and patients. As suggested above, SD has the potential to substantially mitigate the long-lasting ramifications caused by the pay-out of disability payments by insurance companies on behalf of chronically infected HCP and the concomitant increase of premiums on hospitals, clinics, and private enterprises. In addition, SD could reduce or even eliminate the litigations brought on behalf of infected HCP and/or employees working in unsafe environments where needle use is common place, such as in healthcare facilities.

### BRIEF DESCRIPTION OF DRAWINGS

FIG. 1 Perspective view of syringe holder with sheathed needle assembly

FIG. 2 Perspective view of syringe and sheath

FIG. 3 Perspective view of syringe holder with sheath only

FIG. 4A Side cross-sectional view of SD without syringe in engaged position

FIG. 4B Top cross-sectional view of SD showing cam system

FIG. 5 Side cross-sectional view of SD without syringe in disengaged position

FIG. 6 Top view of cam piston

FIG. 7 Side view of cam lever with button

FIG. 8 Side cross-sectional view of cam body and cam system retainer space

FIG. 9 Top view of first cam retainer system

FIG. 10 Top view of second cam retainer system

FIG. 11 Top view of first and second cam retainer systems with cam spring

### DESCRIPTION OF EMBODIMENTS

In one embodiment there is a safety device (SD) for preventing injuries, having a body, a base, an engagement opening/channel (hereafter “opening”), and an engagement system for engaging and disengaging an object placed into the engagement opening, wherein the body, base, engagement opening, and engagement system collectively form the safety device (SD). In another embodiment the safety device has an object specific engagement opening. In another embodiment the object is a sheathed needle, for example in connection with a carpule syringe.

In a further embodiment the engagement system includes a cam system retained within the body that can be manipulated to be engaged and disengaged. In another embodiment the cam system has a first retainer system and a second retainer system. In another embodiment the first and second retainer systems interact with each other.

In another embodiment the engagement system comprises a cam lock, a cam spring, a cam piston, and a cam lever. In a further embodiment the cam lock has a cam engagement face and a cam arm, and the cam lever contains a button. In another embodiment the engagement opening and the first and second retainer systems are contained in the body.

In another embodiment there is a safety device for preventing injuries, primarily to HCP, having a body, a base, an engagement opening, and a cam engagement system for engaging and disengaging an object placed into the engagement opening, wherein the body, base, engagement opening, and cam engagement system form the safety device, and wherein the body contains the engagement opening and cam engagement system. In another embodiment the cam engagement system has a first retainer system and a second retainer system, a cam lock, a cam spring, a cam lever, and a cam piston. In yet another embodiment, there is a cam arm and a cam button. In another embodiment the first and second retainer systems interact with each other. In a further embodiment the base is weighted to secure the safety device in place while in operation and the engagement opening is configured to receive a sheathed needle.

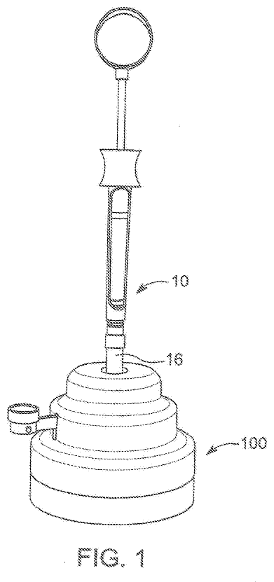

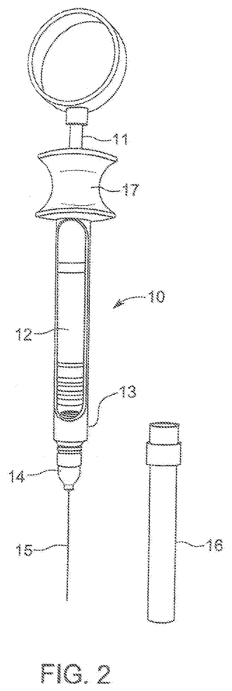

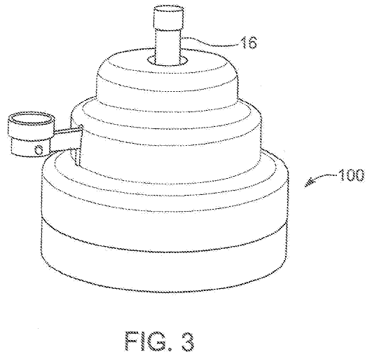

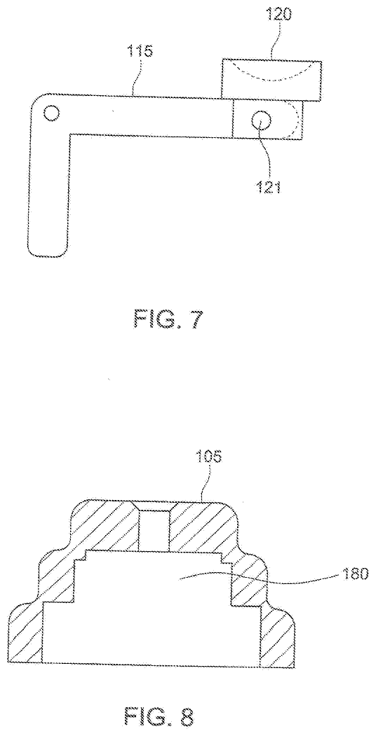

With reference to FIGS. 1-11, the device of the invention will be described in greater detail. In one embodiment, the invention is the safety device (SD) 100 shown in FIG. 1, containing a syringe 10 with a sheathed (16) needle. A typical carpule syringe 10 is shown in detail in FIG. 2. As shown, a typical carpule syringe used in dental offices may contain a plunger 11, carpule 12 containing a liquid anesthetic or other medicament, the needle barrel 13, the hub 14, the needle 15, and handle 17. A needle sheath 16 is shown in FIG. 1 with syringe 10 in the SD 100. FIG. 3 shows the SD 100 containing just the needle sheath 16, for example after the syringe 10 has been removed for use by HCP to treat a patient.

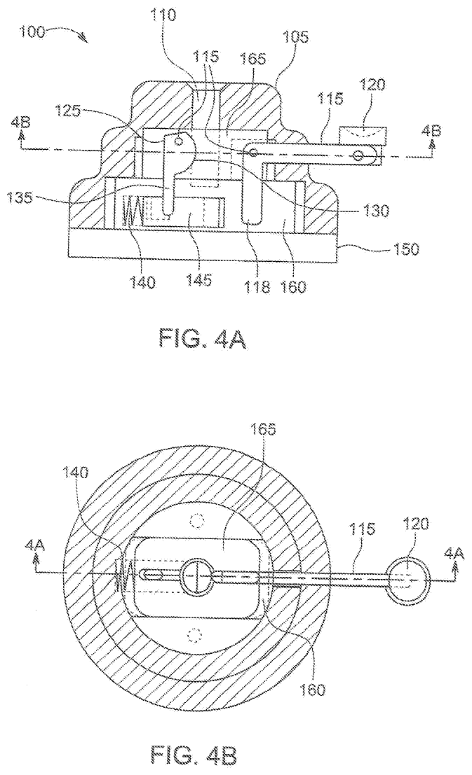

In one embodiment shown in FIG. 4A the SD 100 is shown with a body 105, a needle engagement opening 110, a cam lever 115, cam button 120, cam lock 125, cam engagement face 130, cam arm 135, cam spring 140, cam piston 145, base 150, cam pins 155, first cam retainer 165 and second cam retainer 160. FIG. 4B shows a top cross section view of FIG. 4A, showing a view of the first and second cam retainers (165, 160) and cam spring 140. As further shown in FIGS. 4-5, portions of the cam lock 125 and cam lever 115 (i.e., cam engagement face 130) are in the first cam retainer 165 and portions of the cam lock 125 and cam lever 115 (i.e., cam arm and cam lever arm 118), and cam spring 140 are contained in second cam retainer 160.

In another embodiment, FIG. 4A shows SD 100 in an engaged position (syringe+sheath not shown) with cam lever 115 and cam button 120 in the up position after a syringe 10 with sheathed (16) needle is inserted in the engagement opening 110. The cam engagement face 130 protrudes slightly into the engagement opening 110 to secure the sheath 16 in place during operation of SD 100. FIG. 5 shows SD 100 in a disengaged position (syringe+sheath not shown) with cam lever 115 and cam button 120 in down position, which substantially removes cam engagement face 130 contact with sheath 16, and thus allows removal of sheathed syringe 10 from SD 100.

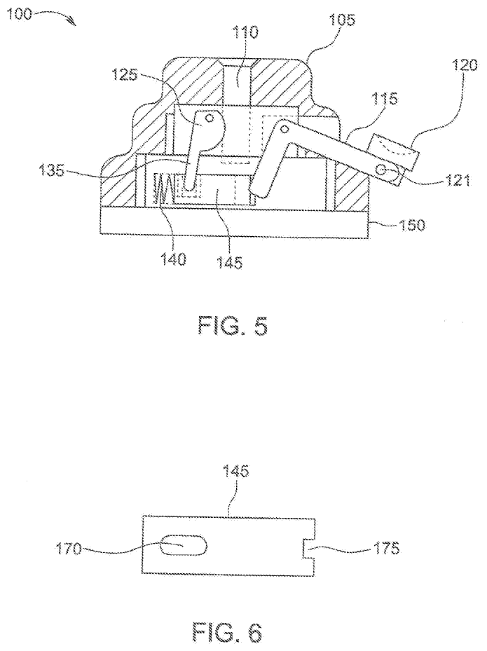

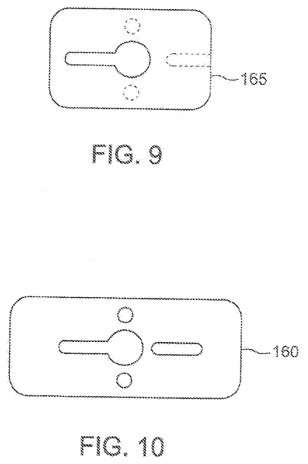

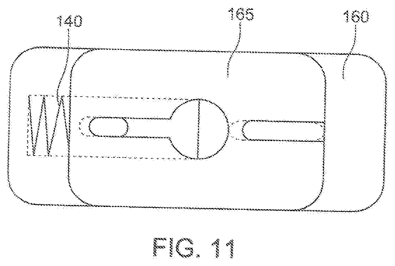

In another embodiment, FIG. 6 shows a top view of cam piston 145, cam lock arm opening 170, and cam lever piston engagement slot 175. In another embodiment, FIG. 8 shows a cam system retainer space 180 in body 105. In another embodiment FIG. 9 shows a top view of a first cam retainer system 165, and FIG. 10 shows a top view of a second cam retainer system 160. In another embodiment FIG. 11 shows a top view of the cam piston system with a cam spring 140 located in the second cam retainer 160, and first cam retainer 165.

In one embodiment, operation of the SD 100 is achieved by inserting a liquid anesthetic/medicament loaded syringe 10 (with sheath 16) into the SD 100 engagement opening 110 with a firm downward pressure using only one hand until the syringe is firmly seated. When the syringe 10 w/sheath 16 is pressed into the opening 110, the cam lock 125 engages the sheath 16 through cam engagement face 130. When HCP is ready to use the syringe 10, she simply pulls the syringe upward, with the sheath 16 staying in the channel 110. After HCP has completed her task with the syringe, syringe 10 w/o sheath is then re-inserted into sheath in opening 110 of SD 100. The thus re-sheathed syringe 10 may then be removed by pushing down on cam button 120 which actuates the cam piston system through engagement of cam lever arm 118 with cam lever engagement slot 175, which in turn moves cam lock arm 135 and compresses cam spring 140. When cam spring 140 is compressed in this manner the cam lock engagement face 130 is substantially removed from the engagement opening 110, thus allowing HCP to easily remove the re-sheathed syringe 10 from the opening 110. HCP may then remove the sheathed needle from syringe 10 and properly dispose of the sheathed needle without incident, for example into a sharps container.

In one embodiment, operation of the SD 100 for use with syringes may be described as follows. Using a single hand, HCP inserts a loaded syringe 10 into the SD engagement opening 110 with a firm downward pressure until syringe is firmly seated in opening. When HCP is ready to use the syringe 10, she pulls upward on syringe handle 17 with one hand, with a slight twisting motion to break any seal, and sheath (cap) will remain securely in place in SD 100 in opening 110. After use of syringe 10 on patient she returns syringe with exposed needle back into sheath 16 in SD 100. To discard a used needle press down cam lever button 120 to release syringe and re-sheathed needle. HCP may then dispose of used needle and sheath in appropriate sharps container. The SD 100 may also be used to hold a fully loaded syringe 10 while HCP awaits next procedure to begin.

In all embodiments described herein the materials selected for use in the manufacture of the safety devices should be durable (i.e., for more than one use) in the environment in which they are operated, and able to withstand sterilization processes and temperatures such as those found in common autoclaves used in the health care industry, for example in dental offices and hospitals. Such materials include but are not limited to metals, alloys, carbon products, and heat resistant polymers.

While embodiments described herein have been developed and described for use in the health care industry, the safety devices described herein may be used in non-health care industries, including for example manufacturing, clean rooms, and laboratories. The scope of the invention described herein is not limited to, but inclusive of, the specific embodiments disclosed herein and those claimed below.

1. A safety device for preventing injuries comprising a body, a base, an engagement opening, and an engagement system for engaging and disengaging an object placed into the engagement opening, wherein the body, base, engagement opening, and engagement system form the safety device.

2. The safety device of claim 1 comprising an engagement opening sized to allow insertion of an object and an engagement system that acts to secure the object until released.

3. The safety device of claim 2 wherein the object is a sheathed needle.

4. The safety device of claim 2 wherein the engagement system comprises a cam system retained within the body that can be manipulated to be engaged and disengaged.

5. The safety device of claim 4 wherein the cam system has a first retainer system and a second retainer system.

6. The safety device of claim 5 wherein the first and second retainer systems interact with each other.

7. The safety device of claim 4 wherein the engagement system comprises a cam lock, a cam spring, a cam piston, and a cam lever.

8. The safety device of claim 7 wherein the cam lock comprises a cam engagement face and a cam arm, and the cam lever comprises a button.

9. The safety device of claim 5 wherein the engagement opening and the first and second retainer systems are contained in the body.

10. A safety device for preventing injuries comprising a body, a base, an engagement opening, and a cam engagement system for engaging and disengaging an object placed into the engagement opening, wherein the body, base, engagement opening, and cam engagement system form the safety device, and wherein the body contains the engagement opening and cam engagement system.

11. The safety device of claim 10 wherein the cam engagement system comprises a first retainer system and a second retainer system, a cam lock, a cam spring, a cam lever, and a cam piston.

12. The safety device of claim 11 further comprising a cam arm and a cam button.

13. The safety device of claim 12 wherein the first and second retainer systems interact with each other.

14. The safety device of claim 13 wherein the base is weighted to secure the safety device in place and the engagement opening is configured to receive a sheathed needle.
